(19) United States
(12) Patent Application Publication
(10) Pub. No.: US 2013/0237795 A1
(43) Pub. Date: Sep. 12, 2013

(54) PERFORMANCE CONCENTRIC ELECTROMYOGRAPHY NEEDLE

(71) Applicant: John Christopher CARR, Surrey (GB)

(72) Inventor: John Christopher CARR, Surrey (GB)

(73) Assignee: CAREFUSION 2200, INC., San Diego, CA (US)

(21) Appl. No.: 13/416,782

(22) Filed: Mar. 9, 2012

(51) Int. Cl. A61B 5/0492

(52) U.S. Cl. 600/395

(57) ABSTRACT

An electromyography needle electrode having an inner core and an outer elongated cannula surrounding the inner core, wherein the inner core includes tungsten. The inner core may comprise at least 99.95 percent tungsten. The outer elongated cannula forms an outer conductive electrode and the inner core forms an inner conductive electrode. At least one insulating layer may be formed between the outer elongated cannula and the inner core. The impedance of the anode and cathode may be matched more closely.

### BACKGROUND

1. Field of Invention

Aspects described herein relate to a needle electrode and an electromyography (EMG) system including a concentric EMG needle having a core comprising tungsten.

2. Description of Related Art

Electromyography (EMG) is a technique for evaluating and recording the electrical activity produced by skeletal muscles. EMG is performed using an instrument called an electromyograph. An electromyography record is called an electromyogram. Such an electromyograph detects the electrical potential generated by muscle cells when these cells are electrically or neurologically activated. EMG signals can be analyzed to detect medical abnormalities and analyze other biomechanical and muscular characteristics.

Human and animal muscle activities are controlled by nerve impulses transmitted electrochemically through the nervous system. Electrical signals related to muscle and nerve activity can be detected through use of medical electrodes applied to the surface of the skin or through electrodes that penetrate the skin, commonly known as needle electrodes. The electrical signals thus detected after suitable amplification may be displayed on an oscilloscope or recorded on a chart recorder or the like, or may be applied to a speaker to provide audio representations of such signals.

Since the electrical signals on the surface of the skin tend to represent a mixing of electrical signals over an undesirably large area, it is often preferable to employ subcutaneously applied needle electrodes to obtain signals from a particular location in the body, and also to obtain the electrical voltage level of the body in general as a reference voltage base. An electromyography system may include a needle electrode for recording electromyographic activity. For example, concentric electromyographic needle electrodes can be used for recording muscular electromyographic activity for the purpose of medical monitoring.

Several problems have been encountered in the use of prior art subcutaneous electrical signal sensing and amplification and display systems. The voltage amplitude of the signals detected by needle electrode sensors can be very low, and in a typical conventional related art system the signal must be conveyed to signal amplifier and recording or display instrument. As the signal can be very small, even small interference, such as that caused by physical movements of the signal carrying electrical cable, can be of sufficient magnitude to mask or at least distort the desired electromyographic signals from the needle sensor.

Thus, the needle electrode needs to be very sensitive to the muscular electrical signal and to avoid influence by outside interference.

Concentric EMG needles include a central electrode and an outer concentric electrode/outer cannula. The inner, central electrode, also referred to interchangeably herein as the “core” or “core wire,” often comprises platinum. Concentric needles typically have a very small surface area for the anode/core and a very large surface are for the cathode/cannula.

Although platinum provides satisfactory qualities, the price of platinum is very expensive and the market for platinum is often volatile. Other less expensive core wires have been used in the industry, e.g. silver or stainless steel; however, these materials can introduce noise of various sorts into the EMG recording. Thus, there is a need in the art for a more affordable core material that provides beneficial EMG recording characteristics in a concentric EMG needle configuration.

### SUMMARY

In light of the above described problems and unmet needs, aspects presented herein include a concentric EMG needle having a core material comprising tungsten. Tungsten is a more affordable material than some materials of the related art and provides enhanced EMG core material characteristics.

Aspects include a needle electrode having an inner core; and an outer elongated cannula surrounding the inner core, wherein the inner core comprises tungsten. The outer elongated cannula forms an outer conductive electrode and the inner core forms an inner conductive electrode. At least one insulating layer may be formed between the outer elongated cannula and the inner core.

In one example implementation, the outer cannula may comprise stainless steel, for example. The inner core may comprise at least at least 99%, e.g., 99.95%, tungsten.

The needle electrode may be an electromyography needle electrode, for example.

Aspects may further include an electromyography system having a controller; an amplifier; and an electrode probe comprising a needle electrode that includes an inner core; and an outer elongated cannula surrounding the inner core, wherein the inner core comprises tungsten.

Additional advantages and novel features of aspects in accordance with the present invention will be set forth in part in the description that follows, and in part will become more apparent to those skilled in the art upon examination of the following or upon learning by practice thereof.

### BRIEF DESCRIPTION OF THE DRAWINGS

Various example illustrations of systems and methods in accordance with aspects of the present invention will be described in detail, with reference to the following figures, wherein:

FIG. 1 illustrates an example needle electrode in accordance with aspects of the present invention.

FIGS. 2A and 2B illustrate an example needle electrode in accordance with aspects of the present invention.

FIGS. 3A and 3B illustrate a table with electrode characteristics for needle electrodes with different core materials, in accordance with aspects of the present invention.

FIGS. 4A, 4B, and 4C illustrate electrode characteristics for needle electrodes with different core materials, in accordance with aspects of the present invention.

FIG. 5 illustrates a table with electrode characteristics for needle electrodes with different core materials, in accordance with aspects of the present invention.

FIG. 6 illustrates a representative diagram of an example EMG system in accordance with aspects of the present invention.

FIG. 7 illustrates a block diagram of an example EMG system in accordance with aspects presented herein.

### DETAILED DESCRIPTION

These and other features and advantages of various aspects of this invention are described in, or are apparent from, the following detailed description of various example implementations.

Intramuscular EMG may involve inserting a needle electrode through the skin into the muscle tissue. A user may observe the electrical activity while inserting the electrode. Such insertional activity may provide valuable information about the state of the muscle and its innervating nerve. Normal muscles at rest make certain, normal electrical signals when the needle is inserted into them. Thus, the electrical activity when the muscle is at rest can also be studied. Abnormal spontaneous activity might indicate some nerve and/or muscle damage. A patient may be asked to contract the muscle smoothly in order to allow the shape, size, and frequency of the resulting motor unit potentials to be judged. Additional analysis can be performed when retracting the electrode a few millimeters, for example, until a group of recordings have been collected. A study may involve an analysis of different skeletal muscles by placing an electrode at various locations.

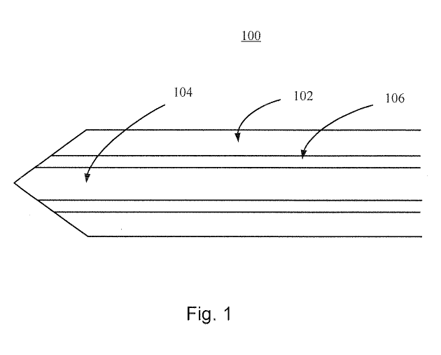

FIG. 1 illustrates an example concentric needle electrode 100 having an outer cannula 102 and an inner core 104. The outer cannula surrounds the inner core except at a tip portion. The outer cannula may comprise stainless steel among other materials, and the inner core may comprise tungsten, for example. In the example electrode 100 of FIG. 1, the outer elongated cannula forms an outer conductive electrode and the inner core forms an inner conductive electrode. At least one insulating layer 106 may be formed between the outer elongated cannula and the inner core. The inner core may comprise at least 99 percent tungsten, or more preferably at least 99.95 percent tungsten, for example.

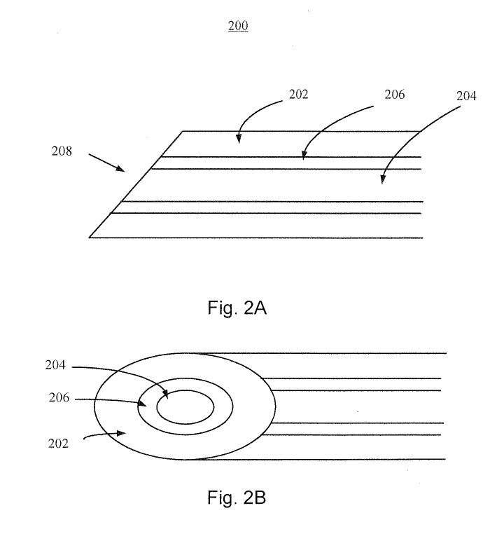

FIGS. 2A and 2B illustrate another example concentric needle electrode 200 having an outer cannula 202, an inner core 204, and a possible insulating layer 206. Similar materials may be used as described in connection with the needle electrode 100 in FIG. 1. FIG. 2A illustrates a side view, and FIG. 2B illustrates an end view showing the surface of the slanted tip 208. Among others, the tip portion of the needle electrode may be slanted, narrowed, beveled, and/or faceted.

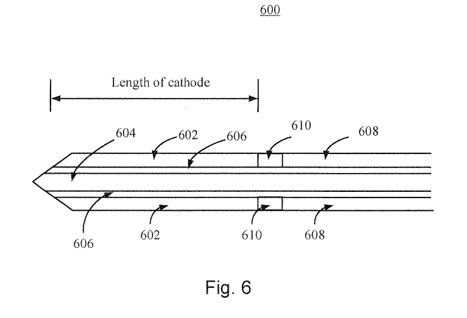

The needle electrode may be an EMG needle electrode that can be used in an EMG system, for example, as illustrated and described further with respect to FIG. 6 below.

As the core/anode material has a very small surface area relative to the patient's body compared to the cannula the noise performance and the impedance of the core to the electrolyte is critical to the performance of the needle. Reducing the impedance of the core to the electrolyte has the effect of improving the common mode rejection ratio (CMRR) of the amplifier and its ability to reject unwanted external interference. The core material should also be resistant to chemical contamination which can produce unwanted electrical noise which produces unwanted artifacts in the EMG recording. The voltage offset of the core material with respect to the cannula is also measured in order to ensure that it is within the range tolerated by a standard EMG amplifier. Thus, a needle electrode that provides a low core impedance, low noise and low offset is important in obtaining a good quality EMG recording.

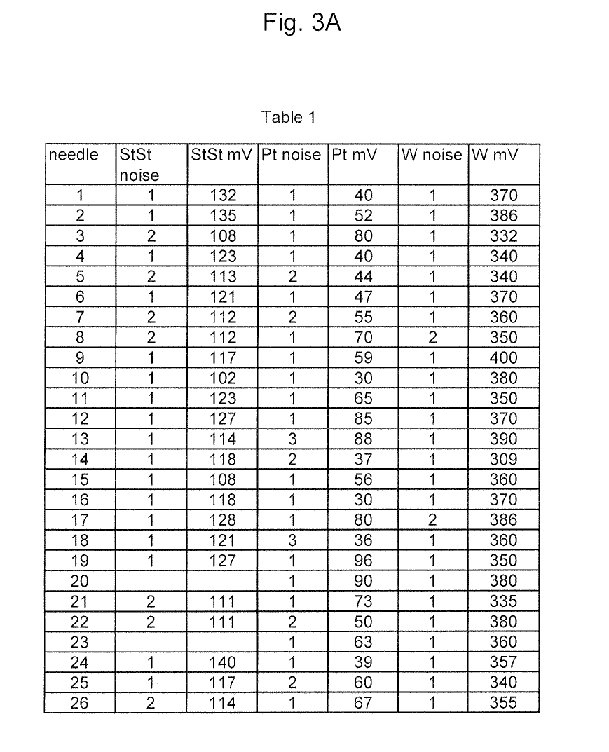

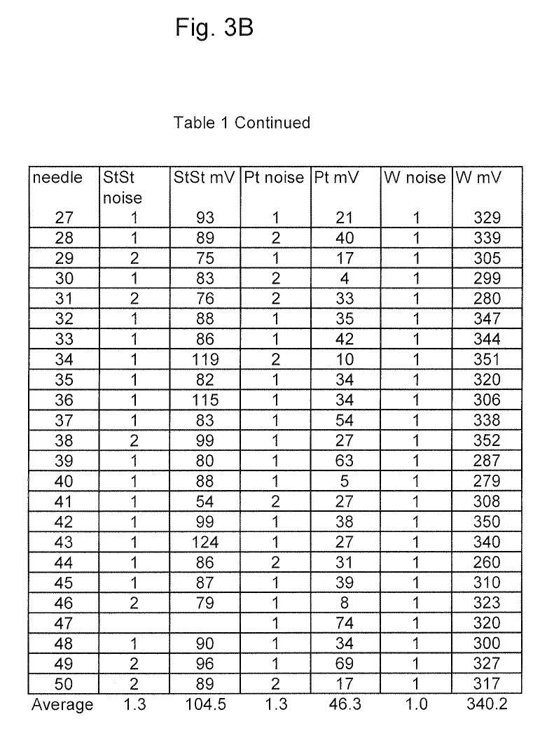

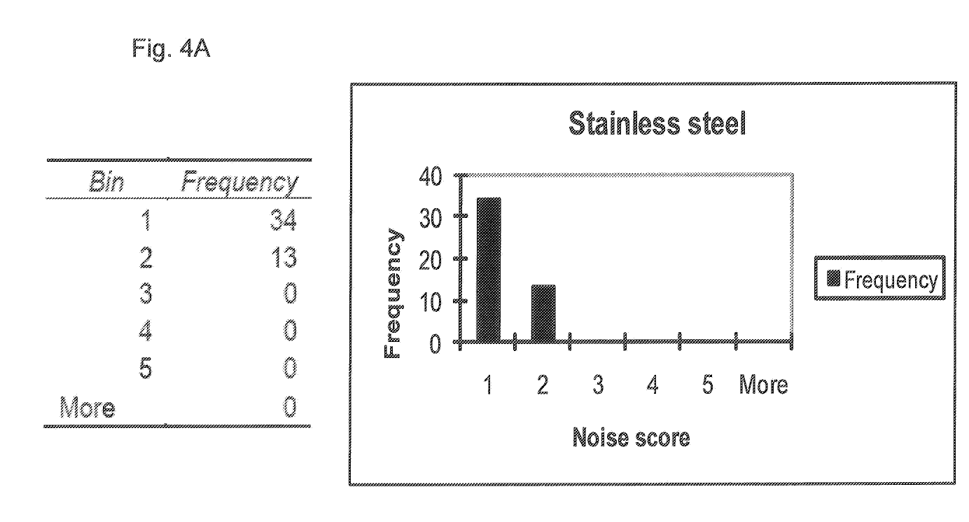

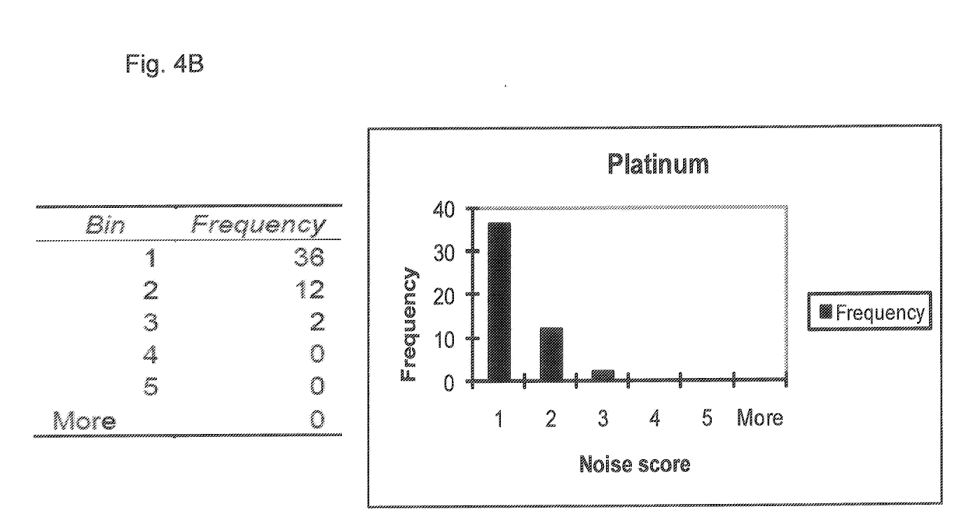

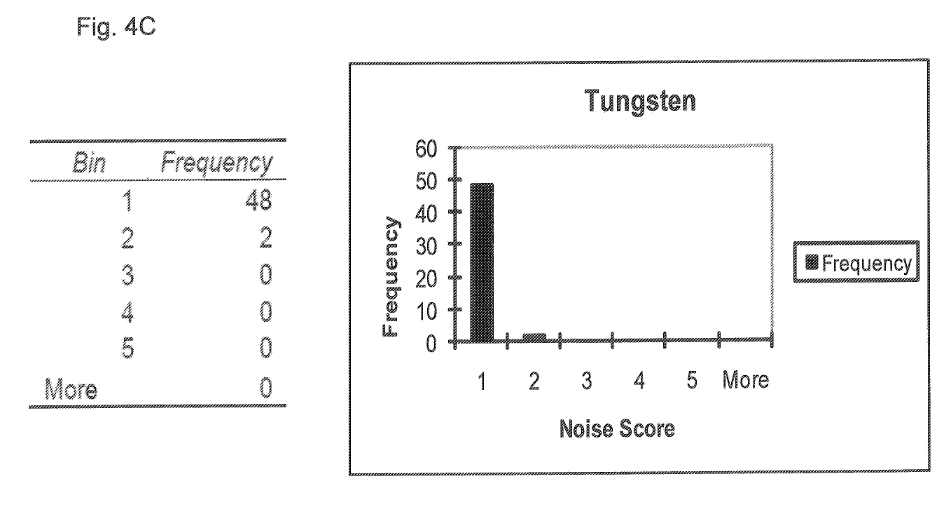

Selective beneficial characteristics of a concentric needle having a tungsten core are illustrated in connection with FIGS. 3A-5. Impedance, noise, and offset results, for example, are shown for concentric EMG needles having various core materials.

In order to assess noise, noise waveforms were recorded on a sample EMG system using an example needle electrode made in accordance with aspects of the present invention. The noise on the needle core wire with respect to the cannula as a reference is assessed. The needle is immersed in normal saline and the following 15 seconds of noise is recorded. The recordings were then ranked using criteria on a scale of 1-5. Level 1 indicates a base line of noise less than 10 μV PkPk. Level 2 indicates all noise being greater than a 100 mS pulse width. Level 3 indicates sporadic pulses of less than 100 mS exceed 50 μV. Level 4 indicates multiple pulses of less than 100 mS that exceed a 50 μV amplitude. Level 5 indicates multiple pulses of less than 100 mS that exceed a 200 μV amplitude. Noise levels 1-3 indicate an acceptable level of noise, whereas levels 4 and 5 indicate an unacceptable level of noise.

The impedance of the recording surface of the various needles cores was also measured when the needle was immersed in saline. The impedance measurement was made against a silver chloride reference electrode. Lower impedance tends to provide improved clarity in the recorded signal and reduced susceptibility to external interference. A lower impedance in the core material reduces the EMG system's susceptibility to both electrostatic and radio frequency interference. The table in FIGS. 3A and 3B illustrates the results for the impedance study.

The offset of the various core materials was also studied. The offset of the metals varied considerably but was negative in all results with respect to the stainless steel needle cannula. The offset was recorded about 1 minute after the needles were immersed in normal saline. The offset of some needles was still increasing at the one minute from immersion time and, as can be seen below, the value varied from needle to needle, as well as among metals. Platinum had the lowest offset at 50 mV. Stainless steel had an offset around 110 mV, and tungsten had an offset of about 340 mV. Offsets of this value should not cause recording problems with standard EMG amplifiers.

The results in FIGS. 3A-5, illustrate that tungsten provides the best overall performance in terms of low noise and low impedance. Tungsten has a higher offset then the other metals relative to stainless steel, but this offset is well within the offset handling capability of EMG amplifiers with which the needles may be used.

Stainless steel has higher noise results than tungsten. However, it is suitable for use as a cannula because the high electrode area reduces the effect of these drawbacks. However, as applied in a needle electrode core, surprisingly, tungsten provides electrode characteristics even beyond those of platinum. In addition, tungsten is a more affordable material with a market that is less volatile than the market for platinum.

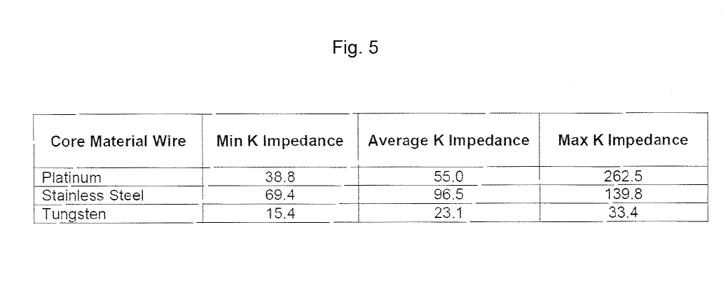

FIG. 5 illustrates a table showing the minimum, average, and maximum impedance in K ohms of the recording surface of 50 needles in accordance with aspects of the present invention at 100 Hz, 10 mV.

Another aspect includes improving the performance and functionality of the EMG needle by reducing the ratio of the impedances of the anode and cathode in order to improve the common mode rejection of the needle. This approach can provide a needle that is more immune to line and RF interference than some other approaches.

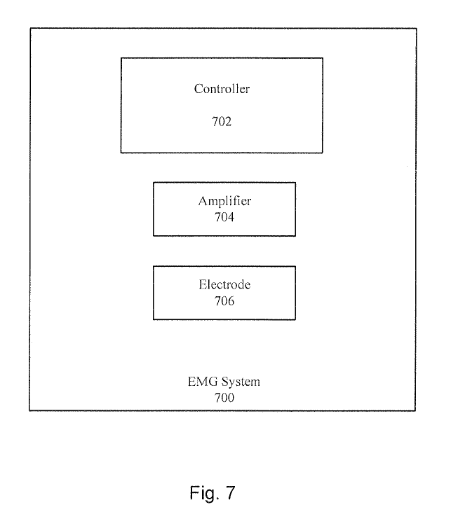

FIG. 7 presents a representative diagram of an example EMG system, in accordance with aspects of the present invention. The EMG system 700 illustrated includes a controller 702 for controlling. An amplifier 704 amplifies the signal received from an electrode 706. The electrode 706 may include any of the aspects of the example needle electrodes described above. As noted above, the amplifier may be susceptible to interference. Thus, it may be important that the needle electrode provide the best signal clarity possible.

Although not illustrated, the EMG system 700 may further include an output device for presenting the electrical recording to a user, such as via visual and/or audio presentation. The system may further include a memory that stores the recording.

While aspects of this invention have been described in conjunction with the example illustrations outlined above, various alternatives, modifications, variations, improvements, and/or substantial equivalents, whether known or that are or may be presently unforeseen, may become apparent to those having at least ordinary skill in the art. Accordingly, the example implementations in accordance with aspects of the invention, as set forth above, are intended to be illustrative, not limiting. Various changes may be made without departing from the spirit and scope hereof. Therefore, aspects of the invention are intended to embrace all known or later-developed alternatives, modifications, variations, improvements, and/or substantial equivalents.

What is claimed is:

1. A needle electrode, comprising:
an inner core; and
an outer elongated cannula surrounding the inner core,
wherein the inner core comprises tungsten.

2. The needle electrode according to claim 1, wherein the outer elongated cannula forms an outer conductive electrode portion and the inner core forms an inner conductive electrode portion.

3. The needle electrode according to claim 2, further comprising:
an insulating layer formed between the outer elongated cannula and the inner core.

4. The needle electrode according to claim 3, wherein the outer cannula comprises stainless steel.

5. The needle electrode according to claim 3, wherein the inner core comprises at least 99.95 percent tungsten.

6. The needle electrode according to claim 5, wherein the needle electrode is an electromyography needle electrode.

7. The needle electrode according to claim 1, wherein the inner core forms an anode, wherein a portion of the outer cannula forms a cathode, and wherein the anode and cathode have a reduced impedance ratio compared to a platinum core.

8. An electromyography system, comprising:
a controller;
an amplifier; and
an electrode probe comprising a needle electrode having:
an inner core; and
an outer elongated cannula surrounding the inner core,
wherein the inner core comprises tungsten.

9. The electromyography system according to claim 8, wherein the outer elongated cannula forms an outer conductive electrode portion and the inner core forms an inner conductive electrode portion.

10. The electromyography system according to claim 9, further comprising:
an insulating layer formed between the outer elongated cannula and the inner core.

11. The electromyography system according to claim 10, wherein the outer cannula comprises stainless steel.

12. The electromyography system according to claim 10, wherein the inner core comprises at least 99.95 percent tungsten.

13. The electromyography system according to claim 8, wherein the inner core forms an anode, wherein a portion of the outer cannula forms a cathode, and wherein the anode and the cathode have a reduced impedance ratio compared to a platinum core.
